(19) United States
(12) Patent Application Publication
(10) Pub. No.: US 2025/0231205 A1
(43) Pub. Date: Jul. 17, 2025

(54) MODIFIED CELLS AS MULTIMODAL STANDARDS FOR CYTOMETRY AND SEPARATION

(71) Applicant: University of Virginia Patent Foundation, Charlottesville, VA (US)

(72) Inventors: Nathan Swami, Charlottesville, VA (US); Carlos Manuel Fernandes Honrado, Macedo de Cavaleiros (PT); Armita Salahi, Charlottesville, VA (US); Mohammad Javad Azizi Jarmoshti, Charlottesville, VA (US)

(21) Appl. No.: 18/730,700

(22) PCT Filed: Jan. 23, 2023
(86) PCT No.: PCT/US2023/061103

§ 371 Date: Jul. 19, 2024

Related U.S. Application Data
(60) Provisional application No. 63/301,544, filed on Jan. 21, 2022.

(51) Int. Cl. G01N 33/96; G01N 15/01; G01N 15/10; G01N 15/1031

(52) U.S. Cl. CPC G01N 33/96; G01N 15/01; G01N 15/1031; G01N 2015/1006; G01N 2015/1028; G01N 2496/05; G01N 2496/25

(57) ABSTRACT

Inline classification of a biological specimen including mammalian cells can include generating an alternating current (AC) electrical stimulus to an electrode structure. The electrode structure can be electrically coupled with a flow cell. A response, elicited by the electrical stimulus, can be received when a model specimen class traverses the flow cell. Using the received response, a corresponding impedance parameter value can be determined, the value indicative of a specified biophysical characteristic corresponding to the model specimen class. The first impedance parameter can be translated to a value corresponding to the specified biophysical characteristic.

### CLAIM OF PRIORITY

This application claims priority to U.S. Provisional Application Ser. No. 63/301,544, filed on Jan. 21, 2022, which is incorporated by reference herein in its entirety, and the benefit of priority of which is claimed herein.

### STATEMENT REGARDING FEDERALLY SPONSORED RESEARCH OR DEVELOPMENT

This invention was made with government support under Grant No. CA044579 awarded by the National Institutes of Health and Grant No. FA2386-18-1-4100 awarded by the Air Force Office of Scientific Research. The government has certain rights in the invention.

### BACKGROUND

Impedance-based cytometry can be used such as to measure electrical properties of cells, sub-cellular bodies, and cellular aggregates. In single-cell impedance cytometry, the detection region can include or use pairs of parallel-facing electrodes, fabricated within a channel. An AC signal can be applied to the top electrodes; and the difference in current flowing through the channel is acquired by the bottom electrodes and measured by detection circuitry. The impedance changes caused by the presence of a particle between the electrode pair are then translated into a change in the current signal being measured, as the current path becomes disturbed. When a particle passes the center of the detection region, individual particle signals are generated. Individual particle signals can be retrieved by signal processing circuitry and, subsequently, are used to plot population distribution and perform data analysis.

### SUMMARY

Biophysical cellular information obtained at single-cell sensitivity can be used within analytical and separation platforms to help associate cell phenotype with markers of disease, infection, and immunity. Certain frequency-modulated, electrically driven microfluidic measurement and separation systems can help enable identification of single cells, e.g., based on biophysical information. For example, such identification can be based on detected biophysical information including, e.g., a cellular size, shape, subcellular membrane morphology, and cytoplasmic organization. Such identification can be challenging, however, in part due to a lack of reliable and reproducible model particles with desirable subcellular electrical phenotypes that can be used as standards to benchmark the electrical physiology of unknown cell types or to benchmark dielectrophoretic separation metrics of novel device strategies. The present inventors have recognized a need for reliable, reproducible model cells for use as multimodal standards, e.g., for use in cytometry and cell separation,

This disclosure relates to systems and methods for using model cells, such as red blood cells (RBCs) or cancer cells, as multimodal standard particles. In an example, the model cells can include systematically modulated subcellular electrophysiology and associated fluorescence level. For example, RBCs can be altered, such as via glutaraldehyde fixation, to vary membrane capacitance of the RBCs. A membrane resealing technique can be applied, e.g., after electrolyte penetration to modulate interior cytoplasmic conductivity and corresponding fluorescence. Similarly, cancer cells can be modified via apoptosis or necrosis to modify a morphology of the cancer cells. The modified RBC cell types or modified cancer cell types can be identified at single-cell sensitivity based on phenomenological impedance metrics. For example, the impedance metrics can be fitted to dielectric models to help estimate or calculate biophysical information. This can enable single-cell impedance data from unknown cell types to be mapped versus model cell types, with known impedance data, for facile determination of subcellular biophysical information and their dielectrophoretic separation conditions. Such an approach can also help reduce a need for certain time-consuming algorithms to estimate biophysical information of an unknown cell type, such as requiring unknown fitting parameters.

This document describes a machine-implemented method for inline classification of a biological specimen including mammalian cells. The machine-implemented method can include triggering generation of an alternating current (AC) electrical stimulus to an electrode structure electrically coupled with a flow cell. For example, the electrical stimulus can be capable of eliciting a detectable response for single-cell detection within the flow cell. A first response elicited by the electrical stimulus can be received, such as when a first analyte including a first model specimen class traverses the flow cell. The received first response can be used to help determine a corresponding first impedance parameter value indicative of a first specified biophysical characteristic corresponding to the first model specimen class. In an example, the first impedance parameter can be translated to a value corresponding to the first specified biophysical characteristic.

An unknown analyte can be classified as corresponding to the first model specimen class based on a value of the first specified biophysical characteristic. For example, the specified biophysical characteristic can include at least one of a cellular size, cellular morphology, conductivity corresponding to cytoplasmic organization, or plasma membrane capacitance. Also, respective responses, elicited by the electrical stimulus when respective different model specimen classes traverse the flow cell, can be received. Here, the respective received responses can be used to help determine corresponding impedance parameter values indicative of respective biophysical characteristics corresponding to the respective different model specimen classes.

A plurality of different model specimen varieties can be characterized, such as by respective corresponding electrical impedance parameters. Here, a plurality of electrical impedance parameters of the target biological specimen can be measured using a specified range of frequencies. The measured electrical impedance parameters of the target biological specimen can be compared with respective electrical impedance parameters of the plurality of different model specimen varieties. For example, at least one distinguishable biophysical feature of the target biological specimen can be determined based on the compared parameters.

First and second individual model specimen varieties and the target biological specimen can be concurrently passed through an assay apparatus. Respective electrical impedance parameters of the first and second individual model specimen varieties can be measured concurrently with the measuring of the plurality of electrical impedance parameters of the target biological specimen.

In an example, the plurality of different model specimen varieties can be characterized or established by modifying a biophysical feature of a first or second mammalian cell varieties towards respective specified metrics. For example, modifying the biophysical feature of at least one of the first or second mammalian cell varieties can include modifying a membrane capacitance of an individual mammalian cell, such as including fixing the individual mammalian cell with glutaraldehyde. Also, modifying the biophysical feature of at least one of the first or second mammalian cell varieties can include modifying an intracellular conductivity of an individual mammalian cell, such as including penetrating the cellular membrane to introduce phosphate buffered saline (PBS) media to the cytoplasm to alter a normalized impedance phase of the individual mammalian cell. Modifying the biophysical feature of at least one of the first or second mammalian cell variety can include causing apoptosis or necrosis to varying intensities (or degrees) to modify a morphology of an individual mammalian cell.

In an example, at least one of the first or second mammalian cell varieties include mammalian red blood cells (RBCs). In an example, at least one of the first or second mammalian cell varieties include mammalian cancer cells. In an example, determining at least one distinguishable biophysical feature of the target biological specimen can be performed without requiring in vitro labeling of the target biological specimen or individual cells included in the plurality of different model specimen varieties. In an example, at least one distinguishable biophysical feature of the target biological specimen can be determined without requiring an optical assay.

The plurality of different model specimen varieties can include an array of model specimen varieties defining respective predetermined electrical impedance parameters across a specified range of frequencies. Measured electrical impedance parameters of the target biological specimen can be compared with respective predetermined electrical impedance parameters of the plurality of different model specimen varieties. For example, this can include plotting the measured electrical impedance parameters of the target biological specimen along the specified range of frequencies. At least one measured electrical impedance parameter can be interpolated along the specified range of frequencies to estimate a biophysical feature between biophysical features defined by two of the array of model specimen varieties. Each of the non-limiting examples described herein can stand on its own, or can be combined in various permutations or combinations with one or more of the other examples.

This Summary is intended to provide an overview of the subject matter of the present patent application. It is not intended to provide an exclusive or exhaustive explanation of the invention. The detailed description is included to provide further information.

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 shows an example biophysical cell classification system.

FIG. 2A depicts an approach to modifying cellular characteristics of red blood cells (RBCs) to establish a plurality of different model cell varieties.

FIG. 2B depicts biophysical differences and impedance metrics between different modified cancer cells.

FIG. 3A depicts an example of translating impedance metrics to cellular biophysical properties.

FIG. 3B depicts an example of translating impedance metrics to cellular biophysical properties.

FIG. 4A depicts an example of a microfluidic device for separating particles in a sample based on biophysical properties.

FIG. 4B depicts an example of a microfluidic device for separating particles in a sample based on biophysical properties.

FIG. 4C depicts an example of a microfluidic device for separating particles in a sample based on biophysical properties.

FIG. 4D is a plot of normal, unaltered RBCs and fixed, model RBCs as obtained via a microfluidic device.

FIG. 5A depicts an example of an electrode configuration for a microfluidic device.

FIG. 5B depicts electrodes arranged on a sidewall of the sensing region.

FIG. 5C depicts an example of an electrode configuration for a microfluidic device.

FIG. 5D is a plot showing particle size as obtained via a microfluidic including a facing electrode configuration.

FIG. 5E is a plot showing particle size as obtained via a microfluidic including a sidewall electrode configuration.

FIG. 6A depicts a microfluidic device including another electrode configuration including three or more electrodes surrounding the sensing region.

FIG. 6B depicts a phase inverted input signal for reducing a noise floor of a microfluidic device.

FIG. 7 is a flowchart that describes a machine-implemented method for biophysical classification of an unknown cell.

FIG. 8 is a flowchart that describes a machine-implemented method for biophysical classification of an unknown cell using a model mammalian cell population.

FIG. 9 is a block diagram illustrating components of a machine.

### DETAILED DESCRIPTION

The present disclosure relates to flow cytometry, and more particularly to using modified mammalian cells as standard particles for quantification and enrichment of a particular cell phenotype during biophysical cytometry. Generally, in flow cytometry, a certain standard particle can be tested with an unknown particle, such as coflowing with the unknown particle during cytometry. Such a standard particle can be characterized by known fluorescence levels, sizes, weights, etc., and used to benchmark the unknown particle during analysis or separation. Examples of standard particles for use in flow cytometry include polystyrene beads or yeast cells.

Microfluidic single-cell electrical measurements by impedance-based flow cytometry and electrically driven separations by dielectrophoresis (DEP) are able to sensitively quantify the cellular biophysical information at high sample throughput (300-500 cells/s) and utilize this through frequency-modulation to distinguish cellular subpopulations. While impedance magnitude at low frequencies (<0.5 MHz) provides highly sensitive size information on each measured cell, the polarization of cell membrane at successively higher frequencies (1-10 MHz) provides information on membrane capacitance, and polarization of the interior at even higher frequencies (>10 MHz) can provide valuable information on cytoplasmic contents, including the nucleus size 10. In this manner, impedance cytometry can be used to quantify subpopulations from heterogeneous samples, including infected red blood cells, activation of various leukocyte subtypes, tumorigenicity of pancreatic cancer cell types, drug sensitivity of cancer cells, bacterial germination from spores, apoptotic bodies generated by drug sensitive cancer cells and to monitor the cell density of spheroids. Also, dielectrophoresis can be applied to isolate cells of a particular phenotype from heterogeneous samples, including circulating tumor cells, stem cell progenitors, cells based on mitochondrial phenotype, bacterial strain discrimination, and isolate secreted exosomes. However, while standard particles with known signal characteristics are used in flow cytometry and fluorescently activated cell sorting for benchmarking each measured cell and to trigger sorting, similar tools are lacking for dielectrophoresis and impedance cytometry. A challenge with using certain standard particles for biophysical cytometry, such as polystyrene beads or yeast cells, is that such particles lack similar cell surface characteristics as a mammalian cell, and therefore do not provide desired accuracy in benchmarking mammalian cells during flow cytometry. The present inventors have recognized a need for reliable, reproducible model cells for use as multimodal standards, e.g., for use in cytometry and cell separation,

This disclosure relates to systems and methods for using model cells, such as red blood cells (RBCs) or cancer cells, as multimodal standard particles. The modified RBC cell types or modified cancer cell types can be identified at single-cell sensitivity based on phenomenological impedance metrics. Such impedance metrics can be fitted to dielectric models to help estimate or calculate biophysical information. This can enable single-cell impedance data from unknown cell types to be mapped versus model cell types, with known impedance data, for estimation of subcellular biophysical information and their dielectrophoretic separation conditions. Such an approach can also help reduce a need for certain time-consuming algorithms to estimate biophysical information of an unknown cell type, such as requiring unknown fitting parameters. Using modified RBCs or modified cancer cells as standard particles can provide higher accuracy and resolution than using other standard particles, such as polystyrene beads or yeast cells.

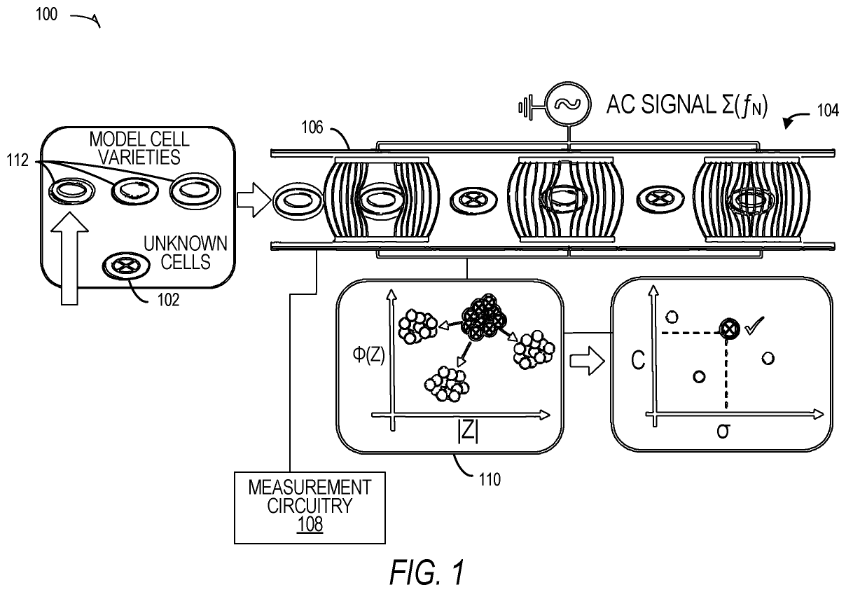

FIG. 1 shows an example biophysical cell classification system 100. The classification system 100 can include or use a biological sample or culture 102, and impedance cytometry device 104 having a test cell 106, measurement circuitry 108 and analysis circuitry 110. As depicted in FIG. 1, the classification system 100 can be used to classify a test cell 106 based on biophysical information, such as impedance metrics. The biological sample or culture 102 can include a sample of unknown cells, such as a sample of mammalian cells, and can include mammalian cell types, such as RBCs, cancer cells, or other mammalian cell types. Herein, unknown relates to cells or cell types that are not yet characterized, e.g., such that impedance metrics of the cells or cell types are not known. The biological sample or culture 102 can be passed through the impedance cytometry device 104, where the impedance metrics of the unknown cells can be measured and compared to the impedance metrics of a standard particle or a model cell.

The impedance cytometry device 104 can include, for example, a microfluidic platform or chamber, such as a microfluidic chip, a flow cytometer, a dielectrophoretic device, or other impedance measurement device. The impedance cytometry device 104 can include measurement circuitry 108, such as electrodes, amplifiers, and other components, to measure impedance metrics associated with the test cell 106. The impedance metrics can include, for example, single-cell capacitance and/or other frequency-dependent impedance metrics, such as an impedance phase angle or an impedance magnitude. The impedance cytometry device 104 can also include analysis circuitry 110, such as a microprocessor, microcontroller, or other system, to analyze the impedance metrics or generate a classification result for the test cell 106.

The impedance cytometry device 104 can measure electrical impedance data of the specimen using a specified range of frequencies, and electrical impedance parameters can be extracted from the electrical impedance data. The electrical impedance parameters can correspond to or characterize biophysical or electrophysiologic features of the specimen. In an example, the impedance parameters can correspond to one or more of electrical size value, cell volume, impedance phase value, impedance magnitude value, or capacitance of constituents comprising the biological specimen.

The unknown cells from the biological sample or culture 102 can flow through the test cell 106 at a specified throughput (e.g., 300-500 cells/s) past microelectrodes under an AC electric field applied over a specified range of frequencies (e.g., 0.5 megahertz (MHz) to 50 MHz). In an example, an impedance of respective detected specimen can be measured by the measurement circuitry 108 concurrently or simultaneously using at least three discrete frequencies: one reference frequency within a range of about 15 MHz and about 20 MHz, and one or more analysis frequencies within a specified analysis frequency range. The reference frequency can be used such as to gate reference particles versus cells or to account for temporal variations within the impedance cytometry device 104. As depicted in FIG. 1, several specified analysis frequency ranges can be used in the classification system 100, such corresponding to respective constituents of the biological specimen. In an example, analysis frequencies less than 1 MHz can be used to measure electrical impedance parameters corresponding to a cell volume.

Analysis frequencies within a range of about 1 MHz to about 10 MHz can correspond to electrical impedance parameters corresponding to a cellular membrane property. Analysis frequencies greater than about 10 MHz can correspond to electrical impedance parameters corresponding to cellular interior properties such an electrophysiology of a nucleus or organelle contained within the specimen. Analysis frequency ranges can be, e.g., from DC or near-DC to about 1 MHz, within a range of about 1 MHz to about 10 MHz, or at a frequency greater than about 10 MHz.

At least one model cell variety 112 can be passed through the test cell 106 concurrently with the unknown cells from the biological sample 102. The at least one model cell variety 112 can include, for example, a modified RBC or modified cancer cell, as discussed herein. The model cell variety 112 can be characterized by known biophysical information, such as impedance metrics, that can be of the same type and range as those measured on the other cells in the sample, such as the unknown cells. The at least one model cell variety 112 can be used to benchmark or calibrate biophysical measurements taken on other cells in the sample.

The analysis circuitry 110 can receive the impedance metrics associated with the unknown cells from the biological sample 102, and can compare the impedance metrics to the impedance metrics associated with the model cell variety 112. The analysis circuitry 110 can classify the unknown cells from the biological sample 102 based on the comparison, such as to classify the unknown cells as a particular cell type or a particular cellular phenotype. The analysis circuitry 110 can also generate a classification result for the unknown cells from the biological sample 102, such as a score or an index that indicates the likelihood that the unknown cells belong to a particular cell type or phenotype. In addition to the model cell varieties, polystyrene beads can be added to samples as an additional reference, such as used for the normalization of magnitude and phase of the impedance signal allowing direct comparison between different populations and or experimental trials.

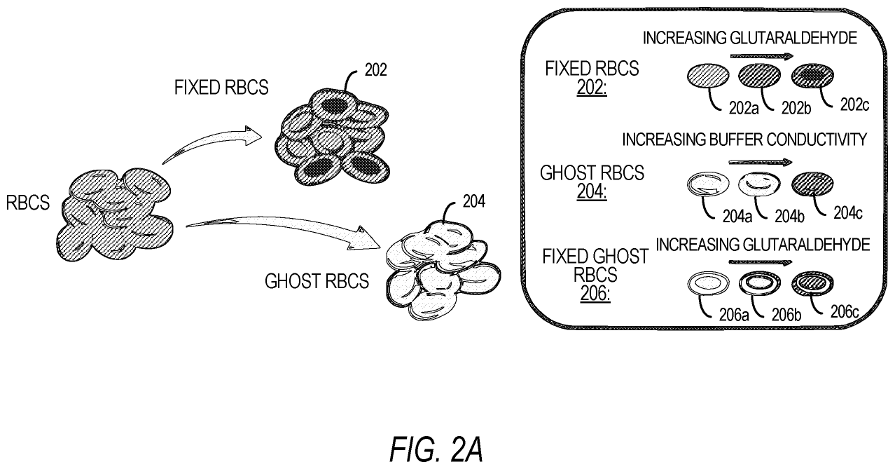

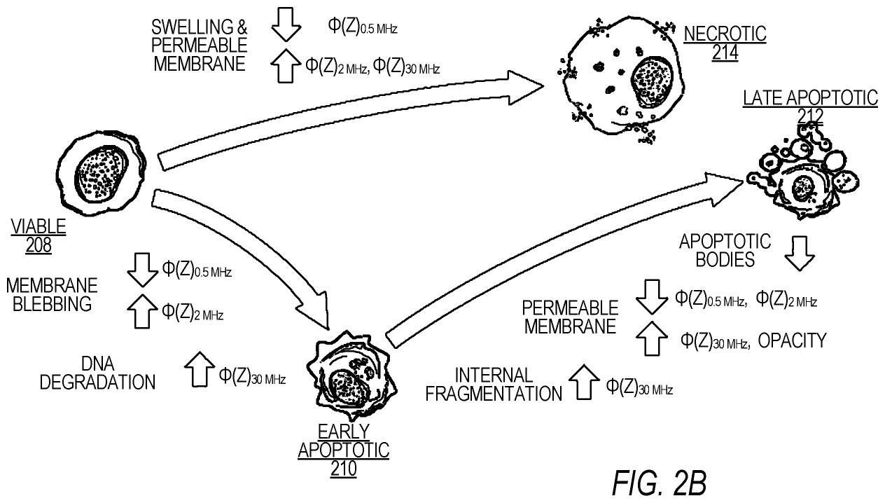

FIG. 2A depicts an approach to modifying cellular characteristics of red blood cells (RBCs) to establish a plurality of different model cell varieties. For example, a biophysical feature of a mammalian cell variety can be modified towards a specified metric, e.g., including modifying a membrane capacitance of an individual mammalian cell. For example, modifying the membrane capacitance can include fixing the individual mammalian cell with glutaraldehyde to define “fixed” RBCs 202. Here, fixing the mammalian cell with glutaraldehyde can cause crosslinking of proteins in the cell membrane and cytoplasm, which can be used to simulate certain pathological states of RBCs. Fixed RBCs 202 can be prepared across a range of increasing glutaraldehyde concentrations or other fixative agents to create a series of cell varieties (202a, 202b, 202c, etc.) with varying respective membrane capacitances.

Presence of the lipid cell membrane can cause mammalian cells to behave as insulators at low frequencies (<1 MHz), enabling an estimation of cell volume. An electrical diameter of an individual mammalian cell can be estimated based on the cube root of impedance magnitude at a frequency level of 0.5 MHz, which is just below that required for cell membrane-induced field dispersion: ∛(|Z|(_{(0.5 MHz)}). At increasing frequencies, the cells can become increasingly conductive due to capacitive coupling across the membrane, until the stabilization of the electric field dispersion at a cutoff frequency, beyond which the field short-circuits the cell membrane (>10 MHz). At a specified frequency range where the membrane capacitance can cause field dispersion (1-10 MHz), an electrical opacity can be estimated or calculated as the impedance magnitude at each probe frequency in this range versus that at 0.5 MHz. In an example where size-controlled insulating polystyrene beads are included to screen the electric field, even at successively higher frequencies, their electrical opacity remains constant at unity. Here, the electrical opacity can be used as a size-normalized impedance metric that varies inversely to the membrane capacitance for comparison versus beads of invariant opacity. Based on the impedance metrics at higher frequencies (>10 MHz) that correspond to properties of the cell interior, there are relatively few significant differences for the respective cell types. This can allow for the ability to generate model particles, such as fixed RBCs 202, with modulated membrane capacitance, but with minimal differences within the cell interior. These fixed RBCs 202 can be provided and characterized as reliable model particles for quantifying functionality of novel DEP designs.

Also, a biophysical feature of a mammalian cell variety can be modified towards a specified metric, e.g., including modifying an intracellular conductivity of an individual mammalian cell to define “ghost” RBCs 204. For example, modifying an intracellular conductivity can include penetrating the cellular membrane to introduce phosphate-buffered saline (PBS) media to the cytoplasm to alter a normalized impedance phase of the individual mammalian cell. Ghost RBCs 204 can be prepared across a range of increasing PBS conductivities to create a series of cell varieties (204a, 204b, 204c, etc.) with varying respective intracellular conductivities.

The varying RC time constant (TRC) arising from an increase in interior conductivity can alter a frequency dispersion in impedance phase (ϕ(Z)), while the impedance phase level would be systematically altered to be shifted further away from insulating beads that are normalized to: ϕ(Z)=0. In an example, membranes of the ghost RBCs can be resealed after penetration, such as in buffers including Fluorescein isothiocyanate (FITC)-dextran at a concentration within a range of about 0.25 milligrams per milliliter (mg/ml) and about 1 mg/ml. Differences between a measured fluorescence level of respective ghost RBC cell varieties (204a, 204b, 204c, etc.) can correspond an FITC level in the buffer during resealing. Hence, by adding differing levels of FITC into the respective penetrating conductive PBS media, each variety of ghost RBCs 204 can be distinguished based on a fluorescence level that is correlated to the ghost RBC 204 variety. For example, ghost RBCs 204 penetrated with media of 1.57 S/m conductivity can be differentiated from those penetrated with media of 2.12 S/m conductivity, simply by using a differing FITC level in the penetrating media (e.g., 0.5 mg/mL for the former and 1 mg/mL for the latter). This ability to independently alter the fluorescence and interior conductivity levels for each ghost RBC type can enable concurrent use such as for distinctions based on their fluorescence or high frequency impedance phase (>10 MHz). Also, these FITC-penetrated ghost RBCs 204 can be characterized by a distinguishable DEP frequency response due to their interior conductivity. Such a distinguishable DEP frequency response can be utilized together with fluorescence imaging or cytometry for facile determination of DEP separation metrics within heterogeneous samples composed of different ghost RBC 204 varieties. As such, ghost RBC modification by membrane resealing to modulate cytoplasm conductivity with minimal alteration in electrical diameter and membrane capacitance can be used to independently modulate their fluorescence level, thereby enabling multimodal identification and optimization of DEP separation strategies to aid in microfluidic device design.

In an example, fixed, ghost RBCs 206 can be prepared across a range of increasing glutaraldehyde concentrations or other fixative agents and prepared across a range of increasing PBS conductivities to create a series of cell varieties (206a, 206b, 206c, etc.) with varying respective intracellular conductivities and varying respective membrane capacitances. Here, the membrane capacitance can be varied for cells of differing interior conductivity. This can be detected based on differing opacity levels (inverse of the membrane capacitance) for ghost RBCs that are first penetrated with first conductivity of PBS media (e.g., 1.57 Siemens per meter (S/m)) and then fixed by differing levels of glutaraldehyde as described above. Fixed, ghost RBCs 206 and fixed RBCs 202 can be distinguished such as within a relatively high-frequency impedance phase response (e.g., >10 MHz).

The impedance spectra from each RBC variety can be fit to standard single-shell dielectric models for computation of their dielectric parameters of cell membrane capacitance and cytoplasmic conductivity, based on predetermined subcellular geometric parameters for RBCs. For example, Table 1 shows examples of dielectric parameters for each RBC type based on fitting of their impedance spectra to a single-shell model. The square bracket indicates glutaraldehyde level for fixation and the rounded bracket indicates conductivity of the penetrating buffer prior to resealing.

| | Membrane capacitance | Cytoplasm conductivity |
|---|---|---|
| | (C_{membrane}) | (σ_{cytoplasm}) |
| Sample | [mF/m²] | [S/m] |
| Control RBCs | 8.85 ± 0.23 | 0.5 ± 0.01 |
| Fixed RBCs | 5.66 ± 0.36 | 0.5 ± 0.03 |
| [0.01%] | | |
| Fixed RBCs | 4.95 ± 0.14 | 0.5 ± 0.01 |
| [0.1%] | | |
| Fixed RBCs [1%] | 4.07 ± 0.25 | 0.5 ± 0.03 |
| Unfixed ghost | 8.85 ± 0.35 | 1.2 ± 0.03 |
| (1.57 S/m) | | |
| Fixed Ghost (1.91 | 7.08 ± 0.22 | 1.4 ± 0.04 |
| S/m) [0.1%] | | |
| Fixed Ghost (2.12 | 7.08 ± 0.35 | 1.6 ± 0.05 |
| S/m) | | |
| [0.1%] | | |
| Fixed ghost | 7.08 ± 0.2 | 1.2 ± 0.06 |
| [0.1%] | | |
| (1.57 S/m) | | |
| Fixed ghost | 6.70 ± 0.18 | 1.2 ± 0.02 |
| [0.3%] | | |
| (1.57 S/m) | | |
| Fixed ghost | 6.20 ± 0.25 | 1.2 ± 0.04 |
| [0.5%] | | |
| (1.57 S/m) | | |

In comparison to control RBCs that are unmodified, the RBCs that are fixed to successively higher levels can exhibit successively lower membrane capacitance and minimal alterations to their cytoplasmic conductivity. Similarly, ghost RBCs penetrated with successively more conductive media, prior to resealing, can exhibit successively higher interior conductivity in comparison to control RBCs that are unmodified.

FIG. 2D depicts biophysical differences and impedance metrics between different modified cancer cells. In a similar fashion to that described above with respect to modified RBCs, an approach to modifying cellular characteristics of mammalian cancer cells can include modifying biophysical features thereof to establish a plurality of different model cell varieties. For example, modifying the biophysical feature of at least one of the first or second mammalian cell variety can include causing apoptosis or necrosis to varying intensities (or degrees) to modify a morphology of an individual mammalian cell. Gemcitabine is a chemotherapeutic drug that can be used to target pancreatic cancer cells, such as to inhibit cellular DNA synthesis and leading to fragmentation of DNA and expression of genes that induce the cell death by apoptosis. Based on impedance-based identification of the four subpopulations (viable 208, early apoptotic 210, late apoptotic 212, and necrotic 214) within the gemcitabine treated pancreatic cancer cell samples, respective the biophysical impedance metrics can be distinguished relevant to their quantification.

For example, a characteristic formation of membrane blebs under apoptosis 210, 212 can increase the surface area of the cell and the capacitance alteration can be related to the increase in membrane permittivity. This increase can increase ΦZ_{2 MHz}, while the onset of apoptotic cell shrinkage can reduce ΦZ_{0.5 MHz}. Moreover, the start of chromatin condensation and DNA fragmentation, together with Ca²⁺ regulated alterations to the endoplasmic reticulum, can increase the conductivity of the cell interior. This can increase ΦZ_{2 MHz}, ΦZ_{18 MHz} and ΦZ_{30 MHz}, assuming an intact plasma membrane for apoptotic cells 210, 212. Furthermore, statistically significant differences in all analyzed metrics are also distinguishable between subpopulations at the early-stage apoptosis 210 versus the late-stage apoptosis 212. Continuation of the internal fragmentation, including nuclear and organelle degradation, can cause lowering of the insulating intracellular material, thereby causing an increase in internal conductivity that is reflected in a significant increase in ΦZ_{18 MHz} and ΦZ_{30 MHz}. Furthermore, with the onset of the “secondary necrosis” onward from late apoptosis 212, the plasma membrane can become progressively permeabilized to lead to alterations similar to those for necrosis, e.g., a significant decrease in both ΦZ_{0.5 MHz} and ΦZ_{2 MHz}, and a significant increase in ΦZ_{18 MHz} and ΦZ_{30 MHz}, due to ion exchange between the intracellular and external media. The transition from early apoptosis 210 to late apoptosis 212 can also lead to the formation and shedding of apoptotic bodies that vary in size, shape and composition during drug-induced cellular disassembly for removing fragmented internal components, which can lead to a sharp decrease in cell size distinguishable by measuring electrical diameter, especially in comparison to the viable cells 208. Comparing the necrotic 214 and late apoptotic 212 subpopulations, while some phenotypic alterations are common, such as the gradual loss of membrane integrity that leads to significant changes in the impedance metrics, there are also key differences between the two states that can be distinguished based on the machine learning strategies. For instance, the formation of membrane blebs and apoptotic bodies within the late apoptotic 212 subpopulation cause differences in electrical diameter and membrane-related metrics (e.g., ΦZ_{0.5 MHz}, ΦZ_{2 MHz} or magnitude opacity) versus the necrotic 214 subpopulation. Similar machine learning approaches to classifying biological subpopulations are described in PCT application No. PCT/US2021/072441, filed on Nov. 16, 2021, and entitled Automated Classification Of Biological Subpopulations Using Impedance Parameters, which is incorporated by reference herein in its entirety, including for its teaching of machine learning techniques for distinguishing subpopulations of cancer cells, which can be used in combination with the model cell benchmarking approaches described in the present disclosure.

In an example, cells in the early apoptotic 210 versus late apoptotic 212 and necrotic states 214 can be distinguished based on the biophysical metrics measured by multifrequency impedance cytometry using modified cancer cells as model particles for benchmarking and quantification. For example, mammalian cancer cells can be placed in a hypotonic solution such as to case apoptosis or necrosis of the cell. Similar to that described above with respect to the model RBCs, the model cancer cells can be passed during cytometry concurrently with an unknown cancer cell population, and one or more biophysical features of the unknown cancer cell population can be identified based on comparison of the unknown cancer cell population with known biophysical characteristics and corresponding electrical parameters.

Machine learning strategies can be used to train for recognition of biophysical metrics from each apoptotic phenotype based on positive controls from hypotonic treatment of the pancreatic tumor cells. Unsupervised learning can enable subpopulation clustering and supervised learning can be applied on gemcitabine treated pancreatic tumor cells such as to enable regression and pattern prediction. In this manner, the relative intensity of onset of apoptosis under gemcitabine treatment can be distinguished for pancreatic tumors of differing gemcitabine sensitivity based on the cell proportions in the viable 208, early apoptotic 210, late apoptotic 212, and necrotic 214 states. In comparison to viable cells 208, those in the early apoptotic state 210 can exhibit lowered electrical diameter levels due to cell shrinkage, lowered impedance phase at low frequency (˜ΦZ_{0.5 MHz}) due to membrane blebbing and a rise in impedance phase at high frequency (˜ΦZ_{30 MHz}) due to alterations at the cell interior, such as Ca²⁺ regulated alterations to the endoplasmic reticulum, chromatin condensation and DNA fragmentation. Late apoptotic cells 212 can exhibit even sharper drops in electrical diameter and impedance phase at low frequency (˜ΦZ_{0.5 MHz}) versus viable 208 and early apoptotic 210 cells, while continuing to exhibit a rise in impedance phase at high frequency (˜ΦZ_{30 MHz}). On the other hand, cells at the necrotic 214 state can be distinguished from all other phenotypic states based on their much higher impedance phase at high frequency (˜ΦZ_{30 MHz}), e.g., based one uncontrolled ion uptake to the cell interior.

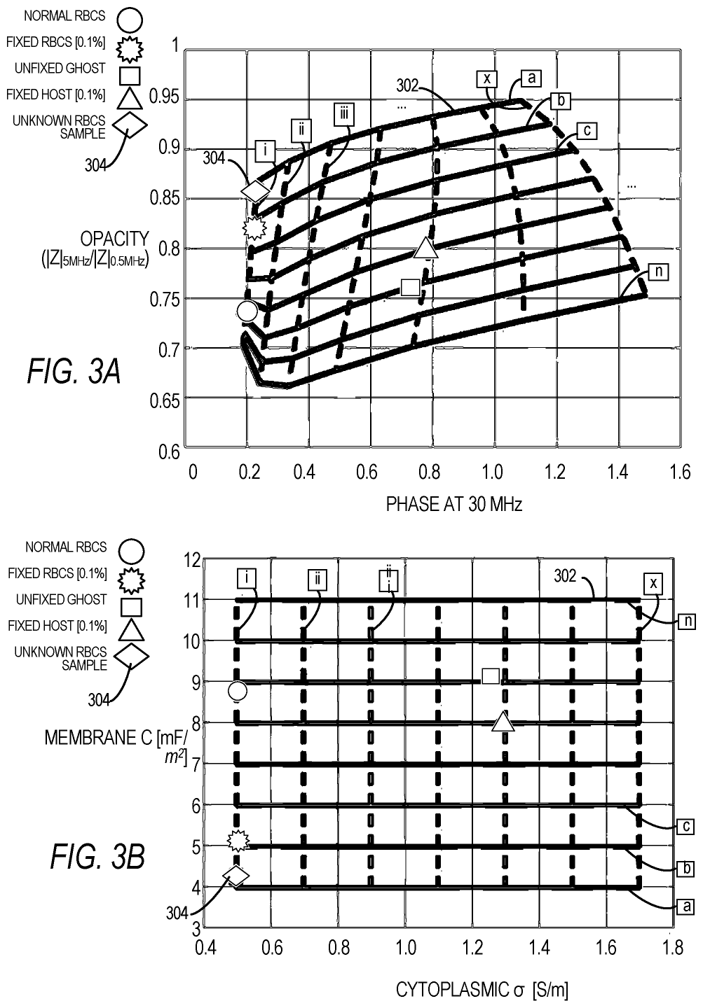

FIG. 3A and FIG. 3B depict an example of translating impedance metrics to cellular biophysical properties. Translating from phenomenological impedance metrics, such as the opacity versus phase-contrast plot in FIG. 3A to cellular biophysical properties of cytoplasm conductivity vs. membrane capacitance in FIG. 3B, as obtained from shell-model fits, can be calculated or estimated for unknown RBCs without the need to perform a shell model fit. Instead, the impedance metrics for the unknown RBCs can be mapped on the respective plot for the model RBCs so that their biophysical properties can be determined by projection onto a subplot 302 visualizing contours of differing membrane capacitance values Here, the dashed lines depict contours of differing cytoplasm conductivity, with each dashed line (i, ii, iii . . . x) indicating a specific level.

This relationship between the phenomenological and biophysical properties for each RBC type is shown in FIG. 3B, wherein the position of each RBC type is plotted with respect to solid lines (a, b, c, . . . n) that indicate varying membrane capacitance levels and the dashed lines (i, ii, iii . . . x) that indicate varying cytoplasmic conductivity levels. Based on this, for an unknown RBC type 304, the impedance opacity at, e.g., ˜5 MHz and the normalized impedance phase at, e.g., ˜30 MHz obtained from the impedance spectra can be located on the map, so that the solid lines (a, b, c, . . . n) and dashed lines (i, ii, iii . . . x) can be used for determining the corresponding cytoplasm conductivity and membrane capacitance values for the unknown RBC type 304 without the need to fit its entire impedance spectra.

Fitting of impedance spectra to dielectric shell models can involve certain fixed parameters (geometric properties like cell size and membrane thickness, for instance) and other “fitting” parameters (e.g., interior permittivity or membrane conductance) to translate from phenomenological impedance metrics to biophysical properties. While these are known for model cells, like the model RBC varieties described herein, the same is not the case for all unknown cell types. Thus, such techniques for translating impedance data into biophysical characteristics, to translate the phenomenological parameters to biophysical properties for unknown cell types can be conducted by simply comparing their phenomenological metrics versus those of the model RBC types, obviating a need for certain geometric properties or certain “fitting” parameters for these unknown cell types. Off-line computation time associated with certain algorithmic fitting approaches is also not needed, thereby allowing for in-line biophysical recognition.

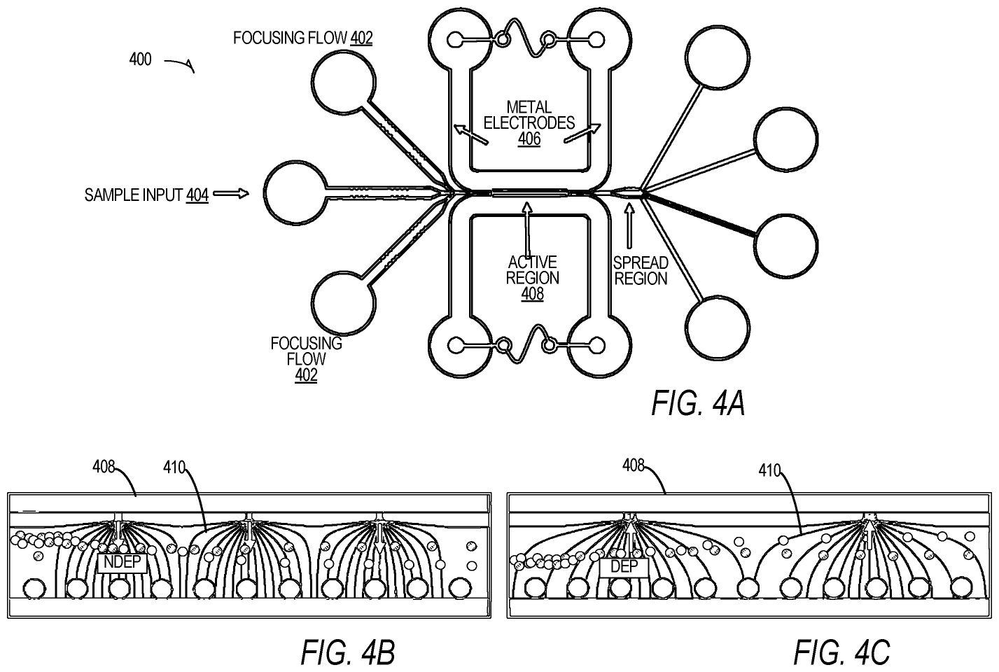

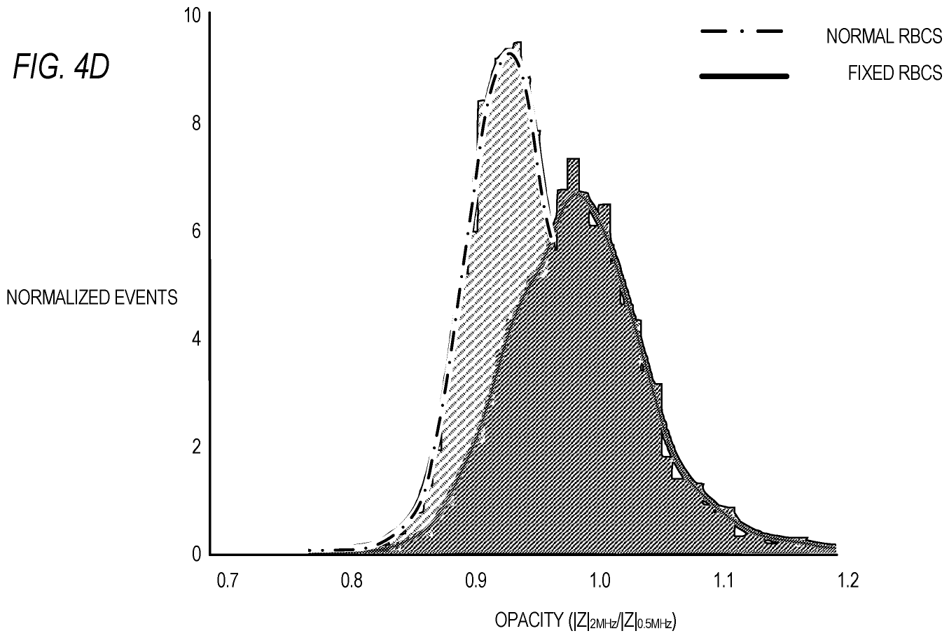

FIG. 4A, FIG. 4B, and FIG. 4C depict an example of a microfluidic device for separating particles in a sample based on biophysical properties. In an example, a microfluidic device 400 can include a focusing flow 402 at or near a sample input 404 to fix its position with respect to the sequential field non-uniformities for separation. Metal electrodes 406 can be included in the device to generate an electric field and a sample can be loaded into the device, resulting in a sample flow. The sample flow can be directed through an active region 408, which can include a series of electrical fields of varying strengths. An individual electric field can cause particles to move toward or away from an individual electrode, resulting in a separation of particles based on their biophysical properties, such as size and/or charge. As depicted in FIG. 4A and FIG. 4B, FIG. 4B electrical fields depicted by field lines 410 can cause particle deflection from initial focused position by negative DEP (nDEP) (FIG. 4A) and positive DEP (FIG. 4B). A sensing system can be included in the device to detect the particles and output a signal. The output signal can be used to adjust the electric field or to control the flow, resulting in a separation of particles based on their biophysical properties.

FIG. 4D is a plot of normal, unaltered RBCs and fixed, model RBCs as obtained via a microfluidic device. As depicted a higher opacity for the collected nDEP fraction (fixed, model RBCs) can be distinguished from the pDEP fraction (normal unaltered RBCs).

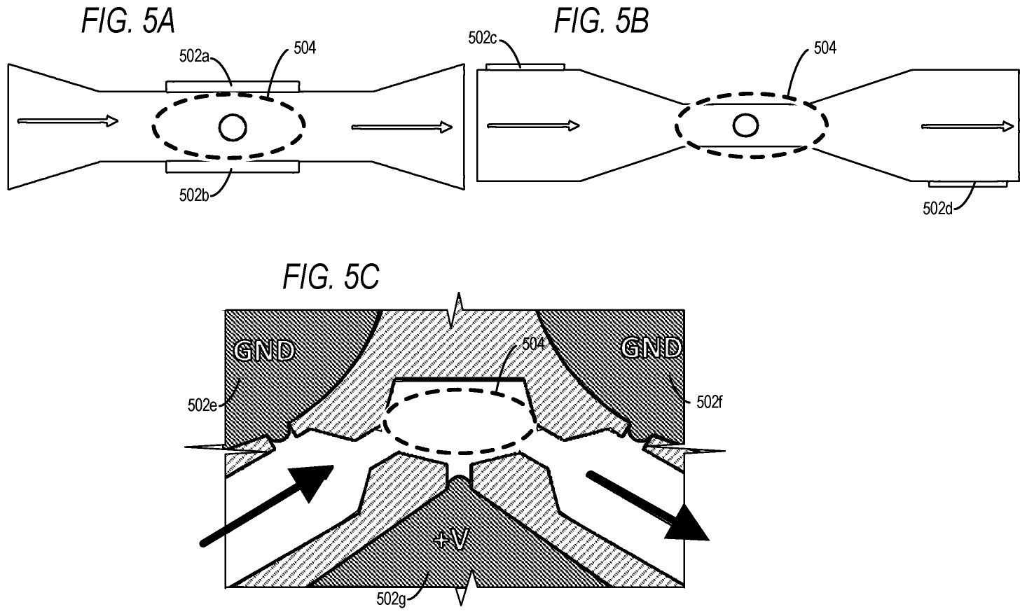

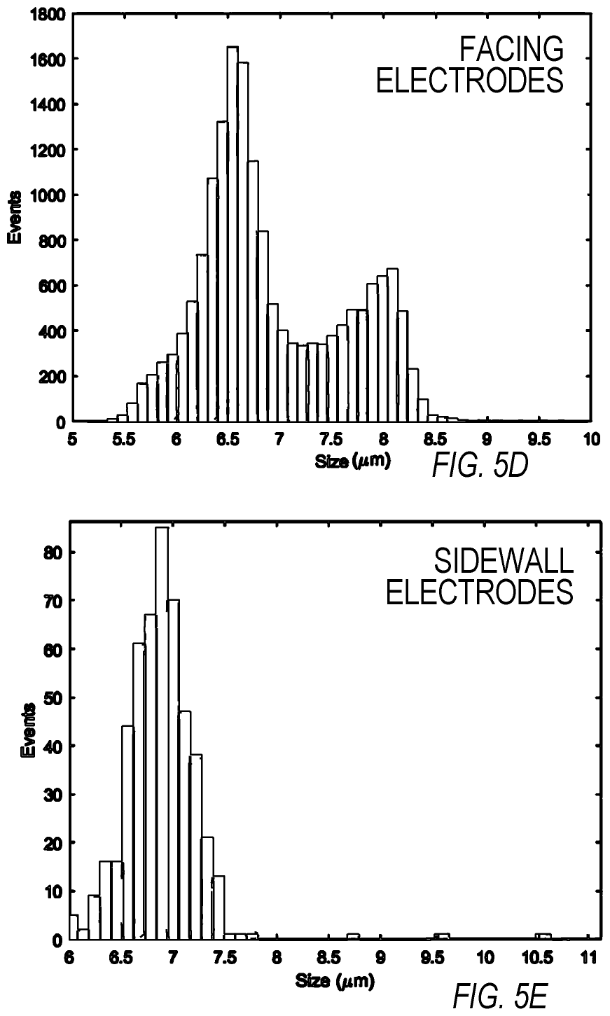

FIG. 5A, FIG. 5B, and FIG. 5C depict examples of electrode configurations for a microfluidic device. As depicted in FIG. 5A, facing planar electrodes 502a and 502b can be positioned facing towards each other on opposite sides of a sensing region 504. FIG. 5B depicts electrodes 502c and 502d arranged on a sidewall of the sensing region 504. For example, the sidewall electrodes 502c and 502d can be offset from the sensing region 504 such that a first electrode 502c is located more proximally to an inlet of a flow cell of the microfluidic device and a second electrode 502d is located more distally to the inlet of the flow cell. Such a sidewall electrode configuration can be used to reduce or eliminate a spread due to different particle positions, such as reducing a tendency for two peaks, such as obtained using facing electrodes, in a plot of particle size per events as shown in FIGS. 5D and 5E. As depicted, the particle size distribution measured using sidewall electrodes (FIG. 5E) is significantly more concentrated than that measured using facing electrodes (FIG. 5D). Such a sidewall configuration can help improve the reproducibility and accuracy of particle size detection of an unknown particle without the need for modeling or algorithmic correction of data to estimate a correct particle size.

Returning to FIG. 5C, a microfluidic device can include an electrode configuration including three or more electrodes (502e, 502f, 502g) surrounding the sensing region 504. For example, the three or more electrodes (502e, 502f, 502g) can include at least one active conductor 502g and at least one reference conductor (502e, 502f) and the conductor 502g can be excited in a differential manner or a passive differential manner.

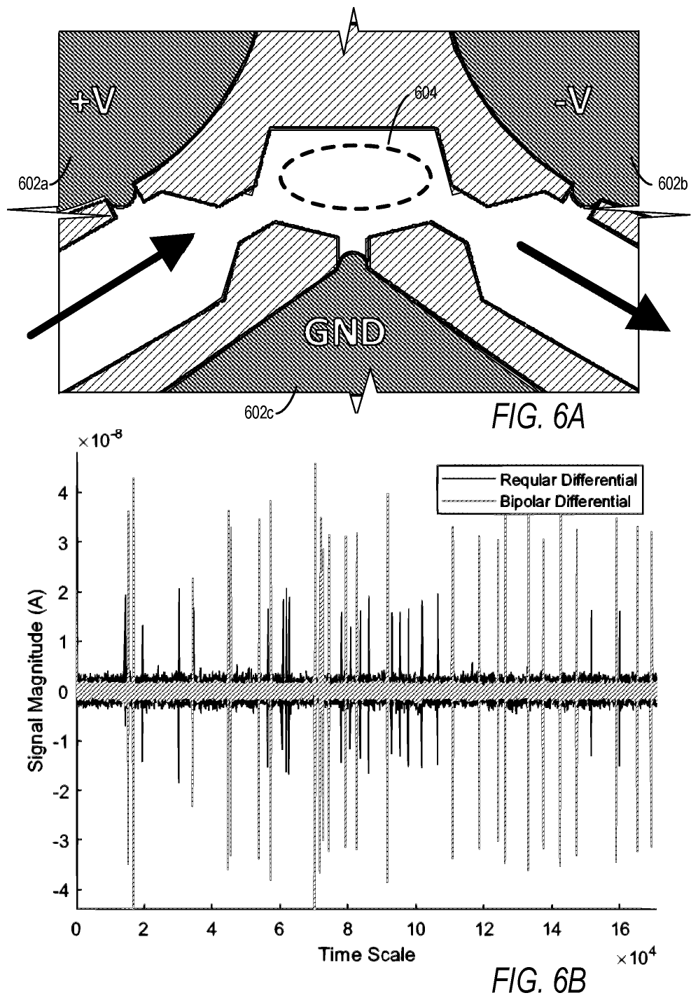

FIG. 6A depicts a microfluidic device including another electrode configuration including three or more electrodes (602a, 602b, 602c) surrounding the sensing region 604. In an example, the three or more electrodes can include at least two active conductors (602a, 602b) and at least one reference conductor (602b), such as to form a bipolar differential including a phase inverted input signal for the excitement of the electrode configuration. As depicted in FIG. 6B, such a phase inverted input signal can reduce a noise floor and enhance gain without introducing signal clipping in absence of particles flowing through a test sell as compared to a regular differential electrode configuration. Such a bipolar differential configuration can also improve a sensitivity over regular differential electrode configurations.

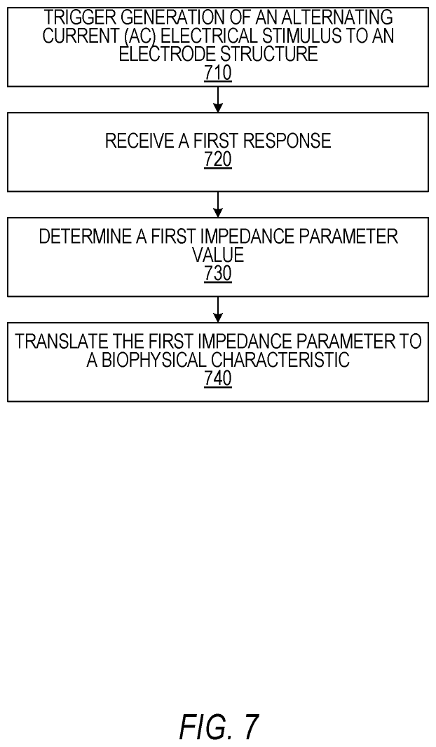

FIG. 7 is a flowchart that describes a machine-implemented method for biophysical classification of an unknown cell.

At 710, the machine-implemented method can include triggering generation of an alternating current (AC) electrical stimulus to an electrode structure electrically coupled with a flow cell. For example, the electrical stimulus can elicit single-cell detection sensitivity within the flow cell.

At 720, the machine-implemented method can include receiving a first response elicited by the electrical stimulus when a first analyte comprising a first model specimen class traverses the flow cell. For example, the machine-implemented method can include receiving respective responses elicited by the electrical stimulus when respective different model specimen classes traverse the flow cell. Such receiving can also include, using the respective received responses, determining corresponding impedance parameter values indicative of respective biophysical characteristics corresponding to the respective different model specimen classes.

At 730, the machine-implemented method can include using the received first response, determining a corresponding first impedance parameter value indicative of a first specified biophysical characteristic corresponding to the first model specimen class.

At 740, the machine-implemented method can include translating the first impedance parameter to a value corresponding to the first specified biophysical characteristic. For example, the machine-implemented method can include classifying an unknown analyte as corresponding to the first model specimen class based on a value of the first specified biophysical characteristic. In an example, the specified biophysical characteristic comprises at least one of a cellular size, cellular morphology, conductivity corresponding to cytoplasmic organization, or plasma membrane capacitance.

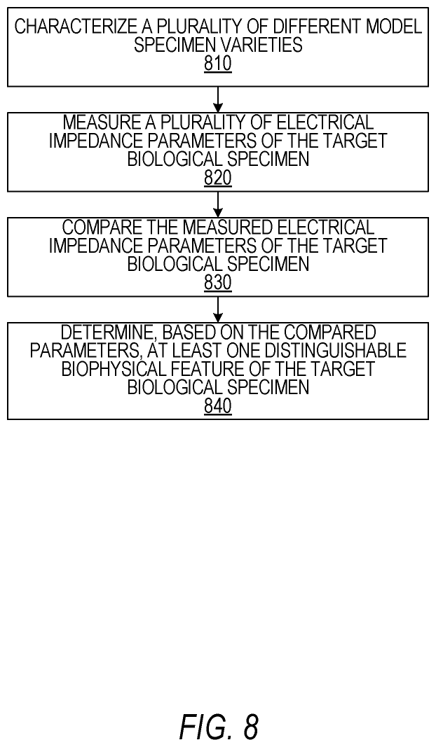

FIG. 8 is a flowchart that describes a machine-implemented method for biophysical classification of an unknown cell using a model mammalian cell population.

In an example, at 810, the machine-implemented method can include characterizing a plurality of different model specimen varieties by respective corresponding electrical impedance parameters. For example, the plurality of different model specimen varieties can include an array of model specimen varieties defining respective predetermined electrical impedance parameters across a specified range of frequencies. In an example, the method can also include passing first and second individual model specimen varieties and the target biological specimen co-flowing through an assay apparatus acquired, the first or second individual model specimen variety and the target biological specimen flowing concurrently with each other.

At 820, the machine-implemented method can include measuring a plurality of electrical impedance parameters of the target biological specimen using a specified range of frequencies. For example, the method can also include measuring, within an assay apparatus, respective electrical impedance parameters of the first and second individual model specimen varieties concurrently with the measuring of the plurality of electrical impedance parameters of the target biological specimen. In an example, determining at least one distinguishable biophysical feature of the target biological specimen can be performed without requiring in vitro labeling of the target biological specimen or individual cells included in the plurality of different model specimen varieties.

At 830, the machine-implemented method can include comparing the measured electrical impedance parameters of the target biological specimen with respective electrical impedance parameters of the plurality of different model specimen varieties. For example, comparing the measured electrical impedance parameters of the target biological specimen with respective predetermined electrical impedance parameters of the plurality of different model specimen varieties can include plotting the measured electrical impedance parameters of the target biological specimen along the specified range of frequencies.

At 840, the machine-implemented method can include determining, based on the compared parameters, at least one distinguishable biophysical feature of the target biological specimen. For example, determining at least one distinguishable biophysical feature of the target biological specimen can do not require an optical assay. Also, determining at least one distinguishable biophysical feature of the target biological specimen can include interpolation of at least one measured electrical impedance parameter along the specified range of frequencies to estimate a biophysical feature between biophysical features defined by two of the array of model specimen varieties. In an example, the machine-implemented method can include causing display of the plotted measured electrical impedance parameters of the target biological specimen along the specified range of frequencies.

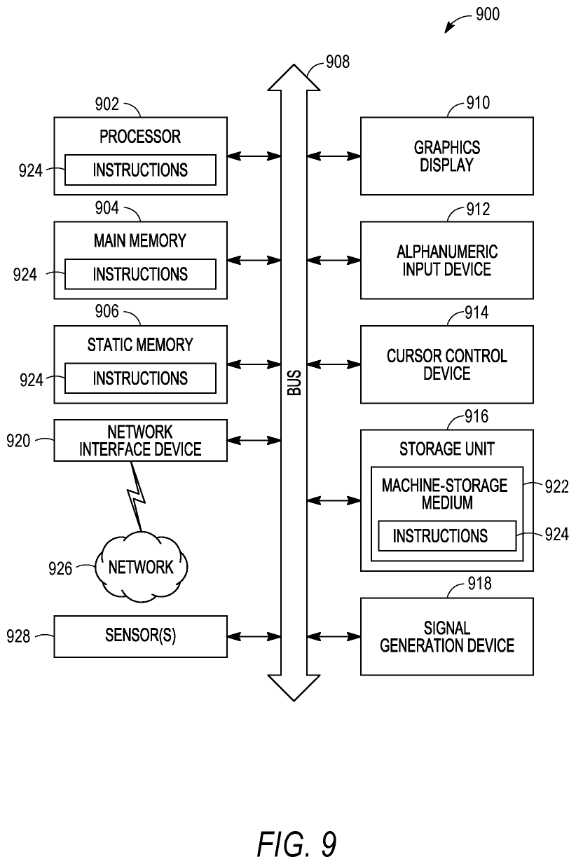

FIG. 9 is a block diagram illustrating components of a machine 900, according to some example embodiments, able to read instructions 924 from a machine-storage medium 922 (e.g., a non-transitory machine-storage medium, a machine-storage medium, a computer-storage medium, or any suitable combination thereof) and perform any one or more of the methodologies discussed herein, in whole or in part. Specifically, FIG. 9 shows the machine 900 in the example form of a computer system (e.g., a computer) within which the instructions 924 (e.g., software, a program, an application, an applet, an app, or other executable code) for causing the machine 900 to perform any one or more of the methodologies discussed herein can be executed, in whole or in part. For example, the instructions 924 can be processor executable instructions that, when executed by a processor of the machine 900, cause the machine 900 to perform the operations outlined above.

In various embodiments, the machine 900 operates as a standalone device or can be communicatively coupled (e.g., networked) to other machines. In a networked deployment, the machine 900 can operate in the capacity of a server machine or a client machine in a server-client network environment, or as a peer machine in a distributed (e.g., peer-to-peer) network environment. The machine 900 can be a server computer, a client computer, a personal computer (PC), a tablet computer, a laptop computer, a netbook, a cellular telephone, a smartphone, a set-top box (STB), a personal digital assistant (PDA), a web appliance, a network router, a network switch, a network bridge, or any machine capable of executing the instructions 924, sequentially or otherwise, that specify actions to be taken by that machine. Further, while only a single machine is illustrated, the term “machine” shall also be taken to include any collection of machines that individually or jointly execute the instructions 924 to perform all or part of any one or more of the methodologies discussed herein.

The machine 900 includes a processor 902 (e.g., a central processing unit (CPU), a graphics processing unit (GPU), a digital signal processor (DSP), an application specific integrated circuit (ASIC), a radio-frequency integrated circuit (RFIC), or any suitable combination thereof), a main memory 904, and a static memory 906, which are configured to communicate with each other via a bus 908. The processor 902 can contain microcircuits that are configurable, temporarily or permanently, by some or all of the instructions 924 such that the processor 902 is configurable to perform any one or more of the methodologies described herein, in whole or in part. For example, a set of one or more microcircuits of the processor 902 can be configurable to execute one or more modules (e.g., software modules) described herein.

The machine 900 can further include a graphics display 910 (e.g., a plasma display panel (PDP), a light emitting diode (LED) display, a liquid crystal display (LCD), a projector, a cathode ray tube (CRT), or any other display capable of displaying graphics or video). The machine 900 can also include an alphanumeric input device 912 (e.g., a keyboard or keypad), a cursor control device 914 (e.g., a mouse, a touchpad, a trackball, a joystick, a motion sensor, an eye tracking device, or other pointing instrument), a storage unit 916, an audio generation device 918 (e.g., a sound card, an amplifier, a speaker, a headphone jack, any suitable combination thereof, or any other suitable signal generation device), and a network interface device 920.

The storage unit 916 includes the machine-storage medium 922 (e.g., a tangible and non-transitory machine-storage medium) on which are stored the instructions 924, embodying any one or more of the methodologies or functions described herein. The instructions 924 can also reside, completely or at least partially, within the main memory 904, within the processor 902 (e.g., within the processor's cache memory), or both, before or during execution thereof by the machine 900. Accordingly, the main memory 904 and the processor 902 can be considered machine-storage media (e.g., tangible and non-transitory machine-storage media). The instructions 924 can be transmitted or received over the network 926 via the network interface device 920. For example, the network interface device 920 can communicate the instructions 924 using any one or more transfer protocols (e.g., Hypertext Transfer Protocol (HTTP)).

In some example embodiments, the machine 900 can be a portable computing device, such as a smart phone or tablet computer, and have one or more additional input components (e.g., sensors 928 or gauges). Examples of the additional input components include an image input component (e.g., one or more cameras), an audio input component (e.g., a microphone), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), and a gas detection component (e.g., a gas sensor). Inputs harvested by any one or more of these input components can be accessible and available for use by any of the modules described herein.

### Executable Instructions and Machine-Storage Medium

The various memories (i.e., 904, 906, and/or memory of the processor(s) 902) and/or storage unit 916 can store one or more sets of instructions and data structures (e.g., software) 924 embodying or utilized by any one or more of the methodologies or functions described herein. These instructions, when executed by processor(s) 902 cause various operations to implement the disclosed embodiments.

As used herein, the terms “machine-storage medium,” “device-storage medium,” “computer-storage medium” (referred to collectively as “machine-storage medium 922”) mean the same thing and can be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media 922 include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms machine-storage medium or media, computer-storage medium or media, and device-storage medium or media 922 specifically exclude carrier waves, modulated data signals, and other such media, at least some of which are covered under the term “signal medium” discussed below. In this context, the machine-storage medium is non-transitory.

### Signal Medium

The term “signal medium” or “transmission medium” shall be taken to include any form of modulated data signal, carrier wave, and so forth. The term “modulated data signal” means a signal that has one or more of its characteristics set or changed in such a matter as to encode information in the signal.

### Computer Readable Medium

The terms “machine-readable medium,” “computer-readable medium” and “device-readable medium” mean the same thing and can be used interchangeably in this disclosure. The terms are defined to include both machine-storage media and signal media. Thus, the terms include both storage devices/media and carrier waves/modulated data signals.

The following, non-limiting examples, detail certain aspects of the present subject matter to solve the challenges and provide the benefits discussed herein, among others.

Aspect 1 is a machine-implemented method for inline classification of a biological specimen comprising mammalian cells, the machine-implemented method comprising: triggering generation of an alternating current (AC) electrical stimulus to an electrode structure electrically coupled with a flow cell, the electrical stimulus configured for single-cell detection sensitivity within the flow cell; receiving a first response elicited by the electrical stimulus when a first analyte comprising a first model specimen class traverses the flow cell; using the received first response, determining a corresponding first impedance parameter value indicative of a first specified biophysical characteristic corresponding to the first model specimen class; and translating the first impedance parameter to a value corresponding to the first specified biophysical characteristic.

In Aspect 2, the subject matter of Aspect 1 includes, classifying an unknown analyte as corresponding to the first model specimen class based on a value of the first specified biophysical characteristic.

In Aspect 3, the subject matter of Aspect 2 includes, wherein the specified biophysical characteristic comprises at least one of a cellular size, cellular morphology, conductivity corresponding to cytoplasmic organization, or plasma membrane capacitance.

In Aspect 4, the subject matter of Aspects 1-3 includes, receiving respective responses elicited by the electrical stimulus when respective different model specimen classes traverse the flow cell; and using the respective received responses, determining corresponding impedance parameter values indicative of respective biophysical characteristics corresponding to the respective different model specimen classes.

In Aspect 5, the subject matter of Aspects 1-4 includes, wherein the electrode structure comprises at least two active conductors and at least one reference conductor.

In Aspect 6, the subject matter of Aspect 5 includes, wherein the at least two active conductors are excited in a differential manner or in a passive differential manner using phase inverted excitement of the electrode structure.

In Aspect 7, the subject matter of Aspects 5-6 includes, wherein the electrodes comprise respective conductors patterned along a sidewall of the flow cell.

In Aspect 8, the subject matter of Aspect 7 includes, wherein a first of the two active conductors is located more proximally to an inlet of the flow cell relative to a second of the two active conductors; and wherein the reference conductor is located between the first and the second of the two active conductors along a flow path through the flow cell.

Aspect 9 is a machine-implemented method for inline classification of biological structures of a target biological specimen, the method comprising: characterizing a plurality of different model specimen varieties by respective corresponding electrical impedance parameters; measuring a plurality of electrical impedance parameters of the target biological specimen using a specified range of frequencies; comparing the measured electrical impedance parameters of the target biological specimen with respective electrical impedance parameters of the plurality of different model specimen varieties; and determining, based on the compared parameters, at least one distinguishable biophysical feature of the target biological specimen.

In Aspect 10, the subject matter of Aspect 9 includes, passing a second individual model specimen variety and the target biological specimen co-flowing through an assay apparatus acquired concurrently with each other; passing a first individual model specimen variety and the target biological specimen through an assay apparatus concurrently with each other.

In Aspect 11, the subject matter of Aspects 9-10 includes, measuring, within an assay apparatus, respective electrical impedance parameters of the first and second individual model specimen varieties concurrently with the measuring of the plurality of electrical impedance parameters of the target biological specimen.

In Aspect 12, the subject matter of Aspects 9-11 includes, providing or obtaining the plurality of different model specimen varieties, including: modifying a biophysical feature of a first mammalian cell variety towards a first specified metric; and modifying a biophysical feature of a second mammalian cell variety towards a second specified metric.

In Aspect 13, the subject matter of Aspect 12 includes, wherein modifying the biophysical feature of at least one of the first or second mammalian cell varieties includes modifying a membrane capacitance of an individual mammalian cell.

In Aspect 14, the subject matter of Aspect 13 includes, wherein modifying the membrane capacitance includes fixing the individual mammalian cell with glutaraldehyde.

In Aspect 15, the subject matter of Aspects 12-14 includes, wherein modifying the biophysical feature of at least one of the first or second mammalian cell varieties includes modifying an intracellular conductivity of an individual mammalian cell.

In Aspect 16, the subject matter of Aspect 15 includes, wherein modifying an intracellular conductivity includes penetrating the cellular membrane to introduce phosphate buffered saline (PBS) media to the cytoplasm to alter a normalized impedance phase of the individual mammalian cell.

In Aspect 17, the subject matter of Aspects 12-16 includes, wherein modifying the biophysical feature of at least one of the first or second mammalian cell variety includes causing apoptosis or necrosis to varying intensities (or degrees) to modify a morphology of an individual mammalian cell.

In Aspect 18, the subject matter of Aspects 12-17 includes, wherein at least one of the first or second mammalian cell varieties include mammalian red blood cells (RBCs).

In Aspect 19, the subject matter of Aspects 12-18 includes, wherein at least one of the first or second mammalian cell varieties include mammalian cancer cells.

In Aspect 20, the subject matter of Aspects 9-19 includes, wherein determining at least one distinguishable biophysical feature of the target biological specimen is performed without requiring in vitro labeling of the target biological specimen or individual cells included in the plurality of different model specimen varieties.

In Aspect 21, the subject matter of Aspects 9-20 includes, wherein determining at least one distinguishable biophysical feature of the target biological specimen does not require an optical assay.

In Aspect 22, the subject matter of Aspects 9-21 includes, wherein the plurality of different model specimen varieties include an array of model specimen varieties defining respective predetermined electrical impedance parameters across a specified range of frequencies.

In Aspect 23, the subject matter of Aspect 22 includes, wherein comparing the measured electrical impedance parameters of the target biological specimen with respective predetermined electrical impedance parameters of the plurality of different model specimen varieties includes plotting the measured electrical impedance parameters of the target biological specimen along the specified range of frequencies.

In Aspect 24, the subject matter of Aspect 23 includes, wherein determining at least one distinguishable biophysical feature of the target biological specimen includes interpolation of at least one measured electrical impedance parameter along the specified range of frequencies to estimate a biophysical feature between biophysical features defined by two of the array of model specimen varieties.

In Aspect 25, the subject matter of Aspects 23-24 includes, causing display of the plotted measured electrical impedance parameters of the target biological specimen along the specified range of frequencies.

Aspect 26 is at least one non-transitory machine-readable medium including instructions for inline classification of biological structures of a target biological specimen, which when deployed or executed by a processor, cause the processor to: characterize a plurality of different model specimen varieties by respective corresponding electrical impedance parameters; measure a plurality of electrical impedance parameters of the target biological specimen using a specified range of frequencies; compare the measured electrical impedance parameters of the target biological specimen with respective electrical impedance parameters of the plurality of different model specimen varieties; and determine, based on the compared parameters, at least one distinguishable biophysical feature of the target biological specimen.

In Aspect 27, the subject matter of Aspect 26 includes instructions which cause the processor to: pass a second individual model specimen variety and the target biological specimen co-flowing through an assay apparatus acquired concurrently with each other; pass a first individual model specimen variety and the target biological specimen through an assay apparatus concurrently with each other.

In Aspect 28, the subject matter of Aspects 26-27 includes instructions which cause the processor to measure, within an assay apparatus, respective electrical impedance parameters of the first and second individual model specimen varieties concurrently with the measuring of the plurality of electrical impedance parameters of the target biological specimen.

In Aspect 29, the subject matter of Aspects 26-28 includes instructions which cause the processor to initiate: modification of a biophysical feature of a first mammalian cell variety towards a first specified metric; and modification of a biophysical feature of a second mammalian cell variety towards a second specified metric.

In Aspect 30, the subject matter of Aspect 29 includes wherein initiating modification of the biophysical feature of at least one of the first or second mammalian cell varieties includes initiating modification of a membrane capacitance of an individual mammalian cell.

In Aspect 31, the subject matter of Aspect 30 includes, wherein initiating modification of the membrane capacitance includes initiating fixing of the individual mammalian cell with glutaraldehyde.

In Aspect 32, the subject matter of Aspects 29-31 includes wherein initiating modification of the biophysical feature of at least one of the first or second mammalian cell varieties includes initiating modification of an intracellular conductivity of an individual mammalian cell.

In Aspect 33, the subject matter of Aspect 32 includes, wherein initiating modification of an intracellular conductivity includes initiating penetration of the cellular membrane to introduce phosphate buffered saline (PBS) media to the cytoplasm to alter a normalized impedance phase of the individual mammalian cell.

In Aspect 34, the subject matter of Aspects 29-33 includes wherein initiating modification of the biophysical feature of at least one of the first or second mammalian cell variety includes causing apoptosis or necrosis to varying intensities (or degrees) to modify a morphology of an individual mammalian cell.

In Aspect 35, the subject matter of Aspects 29-34 includes, wherein at least one of the first or second mammalian cell varieties include mammalian red blood cells (RBCs).

In Aspect 36, the subject matter of Aspects 29-35 includes, wherein at least one of the first or second mammalian cell varieties include mammalian cancer cells.

In Aspect 37, the subject matter of Aspects 26-36 includes, wherein determining at least one distinguishable biophysical feature of the target biological specimen is performed without requiring in vitro labeling of the target biological specimen or individual cells included in the plurality of different model specimen varieties.

In Aspect 38, the subject matter of Aspects 26-37 includes, wherein determining at least one distinguishable biophysical feature of the target biological specimen does not require an optical assay.

In Aspect 39, the subject matter of Aspects 26-38 includes, wherein the plurality of different model specimen varieties include an array of model specimen varieties defining respective predetermined electrical impedance parameters across a specified range of frequencies.

In Aspect 40, the subject matter of Aspect 39 includes, wherein comparing the measured electrical impedance parameters of the target biological specimen with respective predetermined electrical impedance parameters of the plurality of different model specimen varieties includes plotting the measured electrical impedance parameters of the target biological specimen along the specified range of frequencies.

In Aspect 41, the subject matter of Aspect 40 includes, wherein determining at least one distinguishable biophysical feature of the target biological specimen includes interpolating at least one measured electrical impedance parameter along the specified range of frequencies to estimate a biophysical feature between biophysical features defined by two of the array of model specimen varieties.

In Aspect 42, the subject matter of Aspects 40-41 includes instructions which cause the processor to cause display of the plotted measured electrical impedance parameters of the target biological specimen along the specified range of frequencies.

Aspect 43 is at least one machine-readable medium including instructions that, when executed by processing circuitry, cause the processing circuitry to perform operations to implement of any of Aspects 1-42.

Aspect 44 is an apparatus comprising means to implement of any of Aspects 1-42.

Aspect 45 is a system to implement of any of Aspects 1-42.

Aspect 46 is a method to implement of any of Aspects 1-42.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A machine-implemented method for inline classification of a biological specimen comprising mammalian cells, the machine-implemented method comprising:
triggering generation of an alternating current (AC) electrical stimulus to an electrode structure electrically coupled with a flow cell, the electrical stimulus configured for impedance detection at a single-cell sensitivity within a 0.1-100 MHz range within the flow cell;
receiving a first response elicited by the electrical stimulus when a first analyte comprising a first model specimen class traverses the flow cell;
using the received first response, determining a corresponding first impedance parameter value indicative of a first specified biophysical characteristic corresponding to the first model specimen class; and
translating the first impedance parameter to a value corresponding to the first specified biophysical characteristic.

2. The machine-implemented method of claim 1, comprising classifying an unknown analyte as corresponding to the first model specimen class based on a value of the first specified biophysical characteristic.

3. The machine-implemented method of claim 2, wherein the specified biophysical characteristic comprises at least one of a cellular size, cellular morphology, conductivity corresponding to cytoplasmic organization, or plasma membrane capacitance.

4. The machine-implemented method of claim 1, comprising receiving respective responses elicited by the electrical stimulus when respective different model specimen classes traverse the flow cell; and
using the respective received responses, determining corresponding impedance parameter values indicative of respective biophysical characteristics corresponding to the respective different model specimen classes.

5. The machine-implemented method of claim 1, wherein the electrode structure comprises at least two active conductors and at least one reference conductor.

6. The machine-implemented method of claim 5, wherein the at least two active conductors are excited in a differential manner or in a passive differential manner using phase inverted excitement of the electrode structure.

7. The machine-implemented method of claim 5, wherein the electrodes comprise respective conductors patterned along a sidewall of the flow cell.

8. The machine-implemented method of claim 7, wherein a first of the two active conductors is located more proximally to an inlet of the flow cell relative to a second of the two active conductors; and
wherein the reference conductor is located between the first and the second of the two active conductors along a flow path through the flow cell.

9. A machine-implemented method for inline classification of biological structures of a target biological specimen, the method comprising:
characterizing a plurality of different model specimen varieties by respective corresponding electrical impedance parameters;
measuring, within a flow cell structure, a plurality of electrical impedance parameters of the target biological specimen using a specified range of frequencies;
comparing the measured electrical impedance parameters of the target biological specimen with respective electrical impedance parameters of the plurality of different model specimen varieties; and
determining, based on the compared parameters, at least one distinguishable biophysical feature of the target biological specimen; and
establishing or adjusting one or more of a flow parameter or an electrical stimulus parameter in response to the determined at least one distinguishable biophysical feature.

10. The machine-implemented method of claim 9, wherein establishing or adjusting one or more of a flow parameter or an electrical stimulus parameter includes sorting cells or droplets of interest by deflection under dielectrophoresis.

11. The machine-implemented method of claim 9, comprising:
passing a first individual model specimen variety and the target biological specimen co-flowing through an assay apparatus acquired concurrently with each other;
passing a second individual model specimen variety and the target biological specimen through an assay apparatus concurrently with each other.

12. The machine-implemented method of claim 9, comprising measuring, within an assay apparatus, respective electrical impedance parameters of first and second individual model specimen varieties of the plurality of different model specimen varieties concurrently with the measuring of the plurality of electrical impedance parameters of the target biological specimen.

13. The machine-implemented method of claim 9, comprising providing or obtaining the plurality of different model specimen varieties, including:
modifying a biophysical feature of a first mammalian cell variety towards a first specified metric; and
modifying a biophysical feature of a second mammalian cell variety towards a second specified metric.

14. The machine-implemented method of claim 13, wherein modifying the biophysical feature of at least one of the first or second mammalian cell varieties includes modifying a membrane capacitance of an individual mammalian cell.

15. The machine-implemented method of claim 14, wherein modifying the membrane capacitance includes fixing the individual mammalian cell with glutaraldehyde.

16. The machine-implemented method of claim 13, wherein modifying the biophysical feature of at least one of the first or second mammalian cell varieties includes modifying an intracellular conductivity of an individual mammalian cell.

17. The machine-implemented method of claim 16, wherein modifying an intracellular conductivity includes penetrating a cellular membrane to introduce phosphate buffered saline (PBS) media to a cytoplasm to alter a normalized impedance phase of the individual mammalian cell.

18. The machine-implemented method of claim 13, wherein modifying the biophysical feature of at least one of the first or second mammalian cell variety includes causing apoptosis or necrosis to varying intensities (or degrees) to modify a morphology of an individual mammalian cell.

19. (canceled)

20. (canceled)

21. The machine-implemented method of claim 9, wherein determining at least one distinguishable biophysical feature of the target biological specimen is performed without requiring in vitro labeling of the target biological specimen or individual cells included in the plurality of different model specimen varieties.

22. (canceled)

23. (canceled)

24. (canceled)

25. The machine-implemented method of claim 9, wherein:
the plurality of different model specimen varieties include an array of model specimen varieties defining respective predetermined electrical impedance parameters across a specified range of frequencies;
comparing the measured electrical impedance parameters of the target biological specimen with respective predetermined electrical impedance parameters of the plurality of different model specimen varieties includes plotting the measured electrical impedance parameters of the target biological specimen along the specified range of frequencies; and
determining at least one distinguishable biophysical feature of the target biological specimen includes interpolation of at least one measured electrical impedance parameter along the specified range of frequencies to estimate a biophysical feature between biophysical features defined by two of the array of model specimen varieties.

26. The machine-implemented method of claim 25, comprising causing display of the plotted measured electrical impedance parameters of the target biological specimen along the specified range of frequencies.

27. At least one non-transitory machine-readable medium including instructions for inline classification of biological structures of a target biological specimen, which when executed by a processor, cause the processor to:
characterize a plurality of different model specimen varieties by respective corresponding electrical impedance parameters;
measure, within a flow cell structure, a plurality of electrical impedance parameters of the target biological specimen using a specified range of frequencies;
compare the measured electrical impedance parameters of the target biological specimen with respective electrical impedance parameters of the plurality of different model specimen varieties; and
determine, based on the compared parameters, at least one distinguishable biophysical feature of the target biological specimen; and
adjust one or more of a flow parameter or an electrical stimulus parameter in response to the determined at least one distinguishable biophysical feature.

28. The at least one machine-readable medium of claim 27, including instructions which cause the processor to:
pass a first individual model specimen variety and the target biological specimen co-flowing through an assay apparatus acquired concurrently with each other;
pass a second individual model specimen variety and the target biological specimen through an assay apparatus concurrently with each other.

29.-43. (canceled)
